(19) United States
(12) Patent Application Publication
(10) Pub. No.: US 2011/0208102 A1
(43) Pub. Date: Aug. 25, 2011

(54) MEDICAL DEVICE FOR A PUNCTURE SITE OR INFUSION SITE

(75) Inventor: Mokhtar Chawki, Le Vesinet (FR)

(73) Assignee: NEPHROKIT, Le Vesinet (FR)

(21) Appl. No.: 13/121,559

(22) PCT Filed: Sep. 24, 2009
(86) PCT No.: PCT/FR09/01133

§ 371 Date: May 11, 2011

(30) Foreign Application Priority Data
Sep. 30, 2008 (FR) 08/05405

(51) Int. Cl. A61F 13/02

(52) U.S. Cl. 602/54

(57) ABSTRACT

Dressing for a puncture site or infusion site, comprising a first adhesive part (A) and a second part (B) provided with a compress (6), the first part (A) being adhesive on one of its faces, this dressing comprising a third adhesive part (C) on one of its faces that is capable of covering the first part (A), the second part (B) then being placed between the first part (A) and the third part (C), the first part (A) being intended to be attached to the skin at the puncture site or infusion site.

### CROSS REFERENCE TO RELATED APPLICATIONS

This application is a National Stage of International Application No. PCT/FR2009/001133 filed Sep. 24, 2009, which claims priority from French Patent Application No. 08/05405 filed Sep. 30, 2008, the contents of all of which are incorporated herein by reference in their entirety.

### FIELD OF THE INVENTION

The invention relates to the medical or veterinary field.

The invention relates more particularly to a device for protecting a puncture site or infusion site.

The term “puncture” here denotes the operation by means of which a substance, generally liquid, is drained from one part of the body of the human or an animal.

The term “infusion” here denotes the operation by means of which a substance, generally liquid, is introduced into a cavity or into the vessels of the body of a human or an animal.

The expression “puncture site or infusion site” here denotes an area of skin on the body of a human or an animal through which a puncture or infusion device (in particular a needle, a catheter, a cannula) passes.

The invention in particular concerns the medical or veterinary use of a vascular puncture or infusion site. The invention in particular finds an application in the field of haemodialysis, or that of infusions using needles/catheters or short intravenous devices of Cathlon® type (PTFE cannulae).

### BACKGROUND OF THE INVENTION

It is common to withdraw or inject fluids or to administer medicaments to a patient or an animal by means of a tube attached to a needle or a catheter.

One example of puncture is blood collection, common care treatment enabling laboratory examinations on a blood sample withdrawn by venous, capillary or arterial puncture. The hypodermic needle used for the blood collection is often connected to vacuum tubes (for example Vacutainer®, sold by Becton Dickinson).

An example of infusion is intravenous infusion, the common technique of parenteral infusion allowing the dropwise administration of medicaments, of liquids or blood products into the veins, generally a peripheral vein of the upper limb. Intravenous infusion in particular allows the administration of solutes for correcting blood volume.

For the puncture or infusion, a device is inserted through the skin (hypodermic needle, catheter, cannula).

For example, butterfly needles are used for transfusions, blood collections in particular. These needles take their name from the fact that they are provided with a manual pinch zone in the form of butterfly wings. The folding of these wings against one another enables a good grip and facilitates the insertion of the needle under the skin. Once the needle is in place, the wings conventionally come to rest on the skin and an adhesive strip is placed over these wings and over the skin in order to prevent the extraction of the needle. Examples of butterfly needles may be found in the United States patents granted under the following numbers: U.S. Pat. Nos. 2,725,058, 3,064,648, 3,640,275, 4,194,504, 4,300,553, 4,627,842, 5,108,376, 5,149,328, 6,270,480.

It is estimated that around 80% of hospitalized patients are subjected to a treatment administered by intravenous catheter. For certain patients, peripheral catheters are of chronic use. This is the case in particular for patients suffering from acute or chronic renal failure and treated by haemodialysis, or dialysis. The haemodialysis session lasts around four hours, and must be carried out three times a week. Three types of vascular access predominate for haemodialysis: arteriovenous fistulas AVFs, arteriovenous prostheses or grafts, and central venous catheters CVCs. The AVFs are anastomoses created surgically in order to connect an artery and a vein of the patient, commonly in the forearm, or the arm, usually between a radial or humoral artery and its homonymous vein. This anastomosis makes it possible to increase the blood flow within said vein. The creation of an AVF greatly modifies the appearance of the patient's forearm, by creating aneurysmal zones. The needles used for puncturing AVFs are of large calibre, having an internal diameter that typically varies between 1.6 and 2 mm. The needle carrying the patient's blood to the dialysis machine is known as an artery needle, that returning the blood to the patient being known as a vein needle.

When the needle is withdrawn from a blood vessel or from a dialysis fistula, several risks must be taken into account: risk of haemorrhage, risk of blood spray, risk of contamination of medical staff, risk of haematoma, risk of bacterial contamination.

The haemorrhagic risk is very significant for certain patients suffering from acute or chronic renal failure and treated by haemodialysis. During haemodialysis, an anticoagulant is used to limit the risks of clogging of the lumens of capillaries by thrombosis. At the end of the dialysis session, any haemorrhage from the puncture site may prove fatal for the patient.

The risk of blood spray and of contamination is high, the expansion of AIDS and of hepatitides, amongst other blood-borne contagious diseases, only having made this risk more feared. According to the circular by the Délégation générale de la santé [General Health Delegation], dated 20 Apr. 1998 (available at http://www.sante.gouv.fr), relating to the prevention of the transmission of blood-borne infectious agents or biological fluids during treatments in health establishments, a blood exposure accident (BEA) is defined as “any contact with blood or a biological fluid containing blood, comprising either percutaneous exposure or spraying onto a mucous membrane (eye) or onto damaged skin”. The risk of transmission of infectious diseases during a BEA in haemodialysis is currently dominated by the hepatitis C virus due to its prevalence in haemodialysed patients. The risk of HIV transmission after exposure to the blood of a patient carrying HIV is estimated on average at 0.32%. The risk of HBV transmission from an infected patient is very high: between 2% and 40%. This high contagiosity is linked to the very large amount of virus present in the blood and biological fluids (between 1 million to 1 billion viral particles per ml). According to the RAISIN report (monitoring of blood exposure accidents in French health establishments in 2005, available at http://www.invs.sante.fr), the majority of blood exposure accidents occur at the moment a needle is withdrawn through the skin. For haemodialysis sessions on AVF, the moment that is most conducive to the risk of contamination by blood spray is that of the compression, mainly at the moment the needle is withdrawn. Specifically, the haemostatic compress may be imperfectly positioned on the puncture site, in particular for AVFs having a high flow rate or aneurysmal zones.

At the end of a dialysis session, during the disconnection, there is a risk of bacterial contamination generally staphylococcal contamination. This risk is high at the time of the compression of the puncture sites or during a recurrence of bleeding some time after the dialysis session.

The means used in haemodialysis units for overcoming the risks of BEA and of hemorrhage are not standardized. The disconnection protocols are in particular not uniform, several parameters being taken into account: autonomy of the patients, physical capacity of the patients enabling them to be more or less involved in their care, fragility of the skin of, for the most part aged, patients, existence or absence of aneurysmal zones, taking of anticoagulant medicaments of acetylsalicylic acid or anti-vitamin K type or anti-inflammatories. Finally the flow rate of the AVF, and also the presence of hyperpressure via vascular access stenosis may combine to prolong the compression time. In the prior art, several techniques are known that aim to prevent the risks of BEA and of hemorrhage which have just been described.

According to one extremely common technique, a compress, a pad or cotton wool is placed at the puncture site and manual pressure is applied to this compress or this cotton wool, one or two adhesive strips holding the compress or cotton wool in place after the manual pressure is released.

The Applicant has observed that this common technique poses several difficulties.

In dialysed patients bearing an AVF with high blood flow rate, the effect of anticoagulants is such that the bleeding time at the puncture site may extend to 45 minutes after the dialysis session (see document WO 03/099143 page 1, lines 11 to 20). Maintaining pressure on the AVF for such a long time is tedious and tiring. People suffering from neurodegenerative diseases (Parkinson's disease, Parkinsonian syndrome, Alzheimer's disease) may not be able to maintain a manual pressure at the puncture site. The same is true for agitated patients, depressed patients, epileptic patients or patients suffering from chorea or patients struck by convulsions for various reasons.

It is necessary to regularly check the arrest of bleeding at the puncture or infusion site, which requires removing the compress or the cotton wool. This check may give rise to a spray of blood, with risk of contamination of the medical staff, or else a hemorrhage, in particular when the patient is agitated or for dialysed patients having to compress both the venous access and the arterial access of the AVF. Bleeding may take place during the compression of the puncture site, making it necessary to change compress, the presence of blood obscuring the puncture site. Regular verification of the arrest of bleeding by partial or complete removal of the compress may lead to the adhesion of the haemostatic or non-haemostatic compress to the puncture site with risk of detachment of the haemostatic plug.

Document US 2004/0092999 describes an elastic cuff made of latex bonded to which is a hemispherical part made of rubber or latex, this cuff being able to act as a tourniquet before venipuncture and facilitating haemostasis after puncture, the rubber part compressing the puncture site through a compress. The rubber part described in this prior document makes it possible to avoid the application of manual pressure at the puncture site. But the use of this cuff does not make it possible to avoid the risks of blood spray or of hemorrhage when the compressive part is removed to check the puncture site. The dressings described in documents WO 99/08723, US 2005/0256438 have the same drawbacks. The same is true for the pressure dressing for AVF described in document WO 03/099143.

Document WO 2007/044647 describes a pressure dressing that delivers a medicament. The delivery of the medicament is obtained by rupture of a frangible pouch and passage of the medication into a compress covering the skin of the patient. As a variant, the delivery of the medication is obtained by passage through a non-adherent and non-absorbent, air-permeable and water-permeable film, such as a perforated non-woven polyolefin film sold by Delstar under the trade mark Delnet®.

Document U.S. Pat. No. 5,891,074 describes a pressure dressing comprising an absorbent polymer foam placed opposite the puncture or infusion site. The absorbent polymer foam is for example a polyurethane foam sold by Avitar under the trade mark Hydrasorb®. As a variant, a part made of spring steel or made of a polymer material such as polycarbonate, polyethylene, polyurethane is placed in direct contact with the skin. The dressing described in U.S. Pat. No. 5,891,074, like a very large number of dressings proposed in the prior art, targets the automatic application of pressure to the puncture or infusion site, instead of a manual pressure.

Document US 2006/0155235 describes a haemostatic pressure dressing. During the positioning of the dressing, a protective film is removed allowing the release of a haemostatic powder onto the skin of the patient. The powdered haemostatic agent is for example a chitosan. In addition to the haemostatic agents cited in document US 2006/0155235, in the prior art a large number of chemical agents that favour haemostasis are known (based on gelatine, collagen, cellulose oxide, chitosan). Bechtell et al. (Treatment of dialysis access puncture wound bleeding with chitosan dressings, Dialysis & Transplantation, November 2006) describe the use of a haemostatic dressing sold by HemCon, this dressing making it possible to reduce the time of compression on AVFs in order to obtain haemostasis to a few minutes. The dressings proposed by HemCon have been widely used in emergency contexts. These dressings, for example sold under the name HemCon Chito-Flex® contain haemostatic agents (chitosan derivative, see http://www.fda.gov). These dressings have the drawback of obscuring the puncture site that cannot be monitored visually while compressing the site of cutaneous bleeding.

### SUMMARY OF THE INVENTION

The invention aims to propose a device and a process that make it possible to dress a puncture or infusion site, this device not exhibiting the drawbacks of those known previously, and providing a high protection against BEAs, hemorrhages and bacterial contaminations, in particular, but not exclusively, for dialysis vascular accesses.

For these purposes, the invention relates, according to a first aspect, to a dressing for a puncture site or infusion site, comprising a first adhesive part and a second part provided with a compress, the first part being adhesive on one of its faces, this dressing comprising a third adhesive part on one of its faces that is capable of covering the first part, the second part then being placed between the first part and the third part, the first part being intended to be attached to the skin at the puncture site or infusion site.

Advantageously, the first part is formed from a transparent, translucent or semi-transparent material.

Advantageously, the first part is made from a semi-permeable material, for example a micro-perforated material.

In certain embodiments, the first part is formed from a polyethylene strip provided with an acrylic adhesive, in particular provided with perforations having a diameter of less than 0.5 mm, the density of perforations being of the order of one hundred per square centimetre.

In certain embodiments, the first part extends longitudinally, the second part being articulated along an articulation zone longitudinal or transverse to the first part.

In certain embodiments, the third part and the first part extend substantially longitudinally and are articulated to one another, the first part and the third part being articulated longitudinally or transversely to one another.

Advantageously, the third part has a length or surface area substantially equal to or greater than that of the first part.

Advantageously, at least the first part or the third part is made from a material that is elastic, in particular along the two longitudinal and/or transverse directions.

In certain embodiments, the third part is of substantially identical shape to the first part, for example oval, square or rectangular.

The invention relates, according to a second aspect, to an infusion, drain or catheterization kit comprising a dressing as presented above and a needle or cannular or catheter or drain.

The invention relates, according to a third aspect, to the use, as an arteriovenous fistula AVF dressing, of a multilayer comprising:
a first adhesive strip that is transparent and micro-perforated;
an absorbent material;
a second adhesive strip that is articulated to the first adhesive strip.

Advantageously, in a first step, the first adhesive strip is placed at the desired site. In a second step, the absorbent material is applied with pressure against the first adhesive strip. In a third step, the vein or artery puncture needle is completely withdrawn, the pressure being maintained on the absorbent material. In a fourth step, the absorbent material is moved away from the first adhesive strip, allowing a visual control of the haemostasis. In a fifth step, the second adhesive strip is added on top of the absorbent material.

### BRIEF DESCRIPTION OF THE DRAWINGS

Other subjects and advantages of the invention will appear in light of the following description of embodiments, which description will be given with reference to the appended drawings, in which:

FIG. 1 is a perspective view of a patients forearm, provided with an AVF, a butterfly needle being placed in this AVF;

FIG. 2 is a view similar to FIG. 1, showing a first step of the process;

FIG. 3 is a view similar to FIGS. 1 and 2, showing a second step of the process;

FIG. 4 is a view similar to FIGS. 1 to 3, showing a third step of the process;

FIG. 5 is a view similar to FIGS. 1 to 4, showing a fourth step of the process;

FIG. 6 is a view similar to FIGS. 1 to 5, showing a fifth step of the process;

FIG. 7 is a plan view of a dressing, according to one embodiment;

FIG. 8 is a perspective view of the dressing represented in FIG. 7;

FIG. 9 is a plan view of a dressing, according to one embodiment variant;

FIG. 10 is a top view of the dressing from FIG. 9, in an intermediate positioning step;

FIG. 11 is a top view of the dressing from FIG. 9, in a fitting position;

FIG. 12 is a plan view of a first face of a dressing according to another embodiment;

FIG. 13 is a plan view of the second face of the dressing represented in FIG. 12; and

FIG. 14 is a schematic representation of the fitting of the dressing represented in FIGS. 12 and 13, to the forearm of a patient.

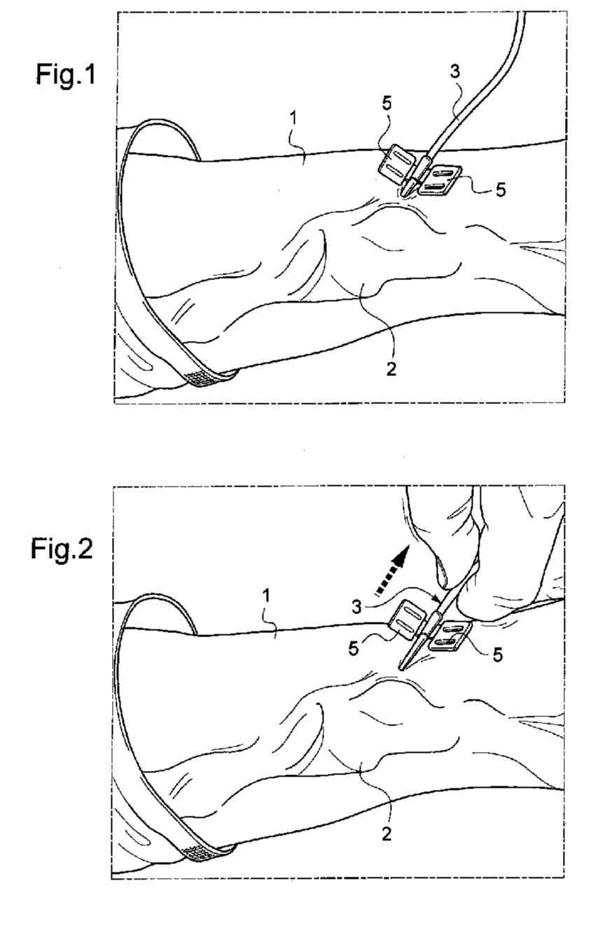

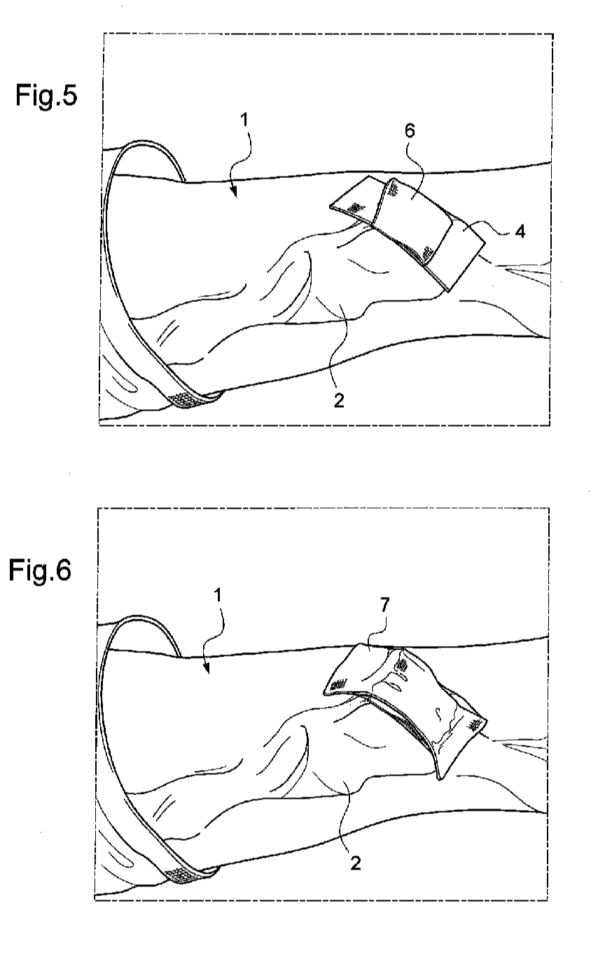

Reference is made to FIGS. 1 to 7.

### DETAILED DESCRIPTION

Represented in FIG. 1 is the forearm 1 of a patient, bearing an AVF. For the purposes of simplification, a single needle is still in place in this AVF. As will be understood by a person skilled in the art, the procedure described in reference to the figures applies successively both to the venous needle and to the arterial needle.

In the appended figures, the needle is of the butterfly needle type. It is however understood that the procedure described also relates to needles lacking the wings of butterfly needles, or else catheters (for example of Cathlon® type) or else cannulae.

The AVF, a vascular access of prime indication that is the most widespread for the dialysed patient, leads to the appearance of aneurysmal zones 2, that can be seen in the figures, the skin being very deformed in the vicinity of the AVF. AVFs are often of short length, so that the two artery and vein needles must be placed close to one another. The use of fastening strips may give rise to a repeated irritation of the skin, promoting excoriations that are particularly sources of bacterial contamination. The fistulas may be kept active for very many years, and the skin of certain dialysed patients is not very supple, thin and fragile due to age.

In a first step, represented in FIG. 2, the needle 3 is slightly withdrawn.

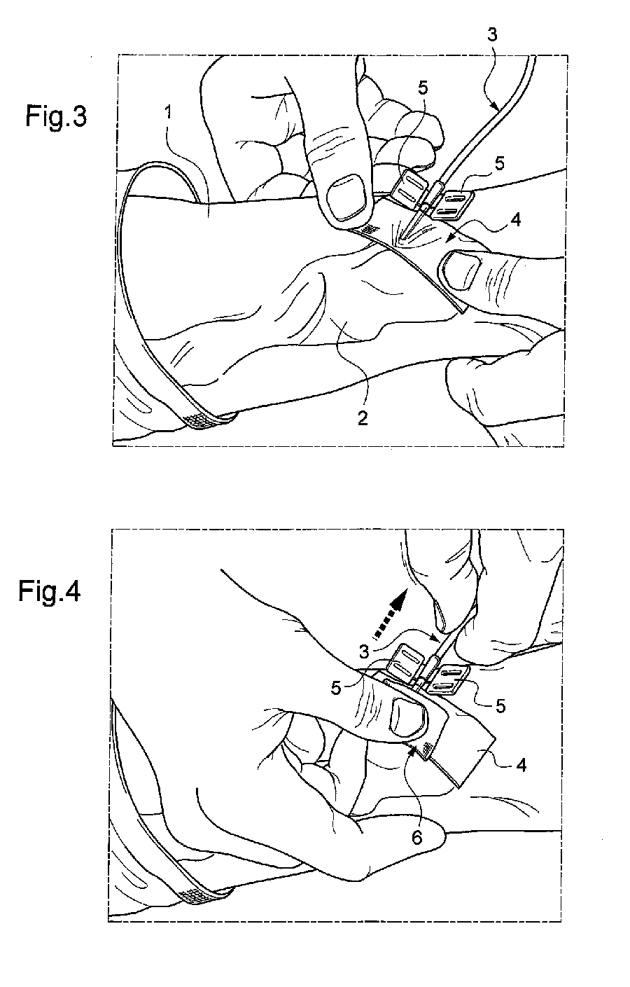

In a second step, represented in FIG. 3, an adhesive strip 4 is placed at the puncture site. This adhesive strip 4 is transparent, semi-transparent or translucent. Advantageously, this adhesive strip 4 is permeable to liquids, for example it is microperforated. Advantageously, this adhesive strip 4 bears haemostatic compounds, for example mixed with the adhesive. It should be noted that the adhesive strip 4 does not stick the needle against the skin of the patient, the wings 5 in particular not being covered by the adhesive strip 4.

In a third step, a compress 6 is applied with pressure against the adhesive strip, level with the puncture site and the needle 3 is completely withdrawn, the pressure being maintained on the compress 6. It should be noted that the adhesive strip is placed between the compress and the skin of the patient.

In a fourth step, the pressure on the compress 6 is released and a visual check of the haemostasis may be carried out. It should be noted that the adhesive strip does not have to be removed, so that the puncture site remains covered during the visual check of the haemostasis. The risks of BEA are thus eliminated, in the same way as the risks of bleeding linked to a removal of the haemostatic plug.

In a fifth step, an adhesive strip 7 is added on top of the compress 6.

The implementation of this process makes it possible to greatly reduce the compression time at the puncture sites of AVFs.

It follows from the experience of the Applicant that the mechanisms resulting in this very significant reduction in the compression time could be the following.

In a first phase, after placement of the adhesive strip covering the puncture site, the blood coming out from the puncture site does not flow under the adhesive strip 4 and remains confined. Before its removal, the needle occupies a space between the skin and the adhesive strip 4, this space possibly forming a short pathway for the blood coming out of the puncture site. This confinement phase has a duration that varies as a function of the hydration state of the whole blood: the more the patient is in hyper-hydration, the longer this first phase is. The duration of this first phase is increased by the taking of anti-vitamin K or in the presence of a platelet anomaly. The duration of this first phase is also increased when the AVF is the site of a stenosis or when the puncturing of the AVF is carried out on an aneurysmal zone or close to the anastomosis. The duration of this first phase may be reduced by exerting a gentle pressure on the anastomosis for around two minutes.

In a second phase, shorter than the first, the blood is screened by its passage through the liquid-permeable adhesive strip 4, and the serum is absorbed by the compress 6. The concentration of formed elements of blood increases in the blood present at the puncture site, due to this screening and this absorption of serum. The viscosity of the blood present at the puncture site increases. The perforations of the adhesive strip 4 are gradually obstructed by viscous blood.

The implementation of the process also makes it possible to greatly reduce the risks of resumption of bleeding.

It follows from the experience of the Applicant that the mechanisms leading to this reduction in the risk of resumption of bleeding could be the following.

The confinement and the screening of the blood flowing from the puncture site results in the creation of small-sized crusts. During the removal of the compress 6, the risk of detachment of these crusts is thus reduced.

The adhesive strip 4, which is semi-permeable, transparent and sterile, protects the vascular puncture site and leaves it visible at any moment without risk of blood spray or of direct bonding of the compress 6 to the puncture site.

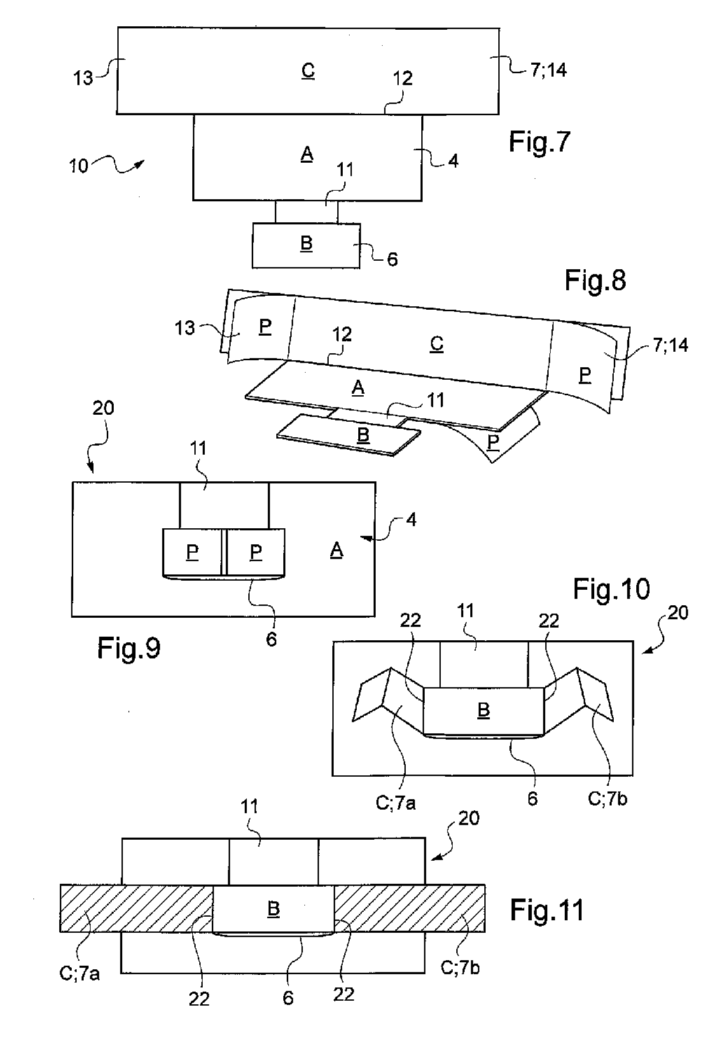

Reference is now made to FIGS. 7 and 8 that illustrate one embodiment of a dressing enabling the use of the process described with reference to FIGS. 1 to 6.

The dressing 10 represented in FIGS. 7 and 8 comprises a first part A forming said adhesive strip 4. Advantageously, a peelable foil covers the adhesive face of this first part A.

The dressing 10 comprises, articulated to this first part A, a second part B forming said compress 6. In the embodiment represented, the second part B is connected to the first part A by a tongue 11. In one embodiment, this tongue 11 is provided with a weakening line that makes it possible to separate the second part B from the first part A of the dressing 10. The expression “weakening line” here denotes any scoring, grooving, perforation, precut or thinning line, the technique used for creating this weakening line being a function, as is known per se, of the type of material used for the dressing. The expression “weakening line” also denotes any mark on the dressing that guides the operator in order to cut the dressing using a tool.

The dressing 10 comprises, articulated to the first part A, a third part C forming said adhesive strip 7. Advantageously, a peelable foil P covers, before use, the adhesive face of the third part C. In the embodiment represented, the third part C is articulated to the first part A by a line of articulation 12 that is substantially longitudinal, that is to say that extends along the largest dimension of the first part A. In one embodiment, this line of articulation 12 is a weakening line within the meaning defined above, enabling the separation of the third part C from the first part A of the dressing 10.

In one embodiment, the compress 6 is at least partly surmounted by a semi-rigid and transparent sheet, for example made of polycarbonate or PET. This sheet participates in the compression of the puncture site, when the adhesive strip 7 is placed under tension against the compress 6.

Advantageously, the adhesive strip 4 formed by the first part A of the dressing is semi-permeable and allows the diffusion of at least certain compounds of the blood from the puncture site to the compress 6.

In one advantageous embodiment, the dressing, except for the compress 6, is made from flexible polymer material or coated fabric. The dressing, except for the compress, is advantageously entirely transparent, translucent or semi-transparent.

The flexibility of the dressing makes it possible to follow the contours of the skin, including in the vicinity of the aneurysmal zones of AVFs. Advantageously, the material constituting the lateral parts 13, 14 of the third part C and where appropriate the entire dressing 10, can be extended along at least one longitudinal direction and more advantageously still along the two longitudinal and transverse directions. The dressing is thus even more adaptable to the various curves of the patient's body.

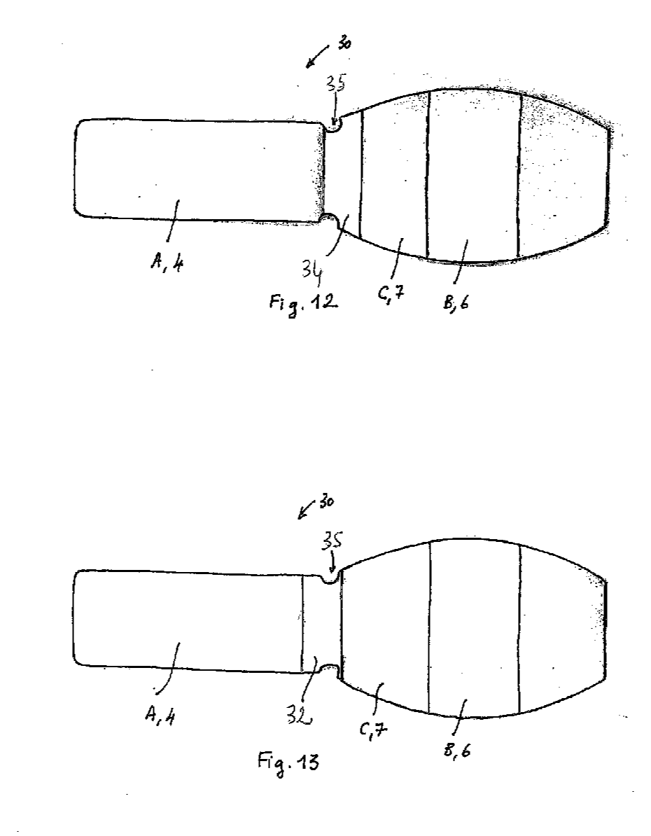

Reference is now made to FIGS. 9 to 11.

The dressing 20 represented in FIGS. 9 to 11 comprises a first part A forming said adhesive strip 4. Advantageously, a peelable foil covers the adhesive face of this first part A.

The dressing 20 comprises, articulated to this first part A, a second part B forming said compress 6. In the embodiment represented, the second part B is connected to the first part A by a tongue 11. In one embodiment, this tongue 11 is provided with a weakening line that makes it possible to separate the second part B from the first part A of the dressing 20. The expression “weakening line” here denotes any scoring, grooving, perforation, precut or thinning line, the technique used for creating this weakening line being a function, as is known per se, of the type of material used for the dressing. The expression “weakening line” also denotes any mark on the dressing that guides the operator in order to cut the dressing using a tool.

The dressing 20 comprises, articulated to the first part A, a third part C forming said adhesive strip 7. Advantageously, a peelable foil P covers, before use, the adhesive face of the third part C.

In the embodiment represented, the third part C forms two tabs 7a, 7b articulated to the second part B by lines of articulation 22 that are substantially transverse, that is to say that extend along the smallest dimension of the first part A. In one embodiment, these lines of articulation 22 are weakening lines within the meaning defined above, allowing the separation of the third part C from the first part A of the dressing 20.

In one embodiment, the compress 6 is at least partly surmounted by a semi-rigid and transparent sheet, for example made of polycarbonate or PET. This sheet participates in the compression of the puncture site, when the adhesive strips 7a, 7b are placed under tension against the compress 6.

Advantageously, the adhesive strip 4 formed by the first part A of the dressing is semi-permeable and allows the diffusion of at least certain compounds of the blood from the puncture site to the compress 6.

In one advantageous embodiment, the dressing, except for the compress 6, is made from flexible polymer material or coated fabric. The dressing, except for the compress, is advantageously entirely transparent, translucent or semi-transparent.

The flexibility of the dressing makes it possible to follow the contours of the skin, including in the vicinity of the aneurysmal zones of AVFs. Advantageously, the material constituting the lateral parts 7a, 7b of the third part C and where appropriate the entire dressing 20, can be extended along at least one longitudinal direction and more advantageously still along the two longitudinal and transverse directions. The dressing is thus even more adaptable to the various curves of the patient's body.

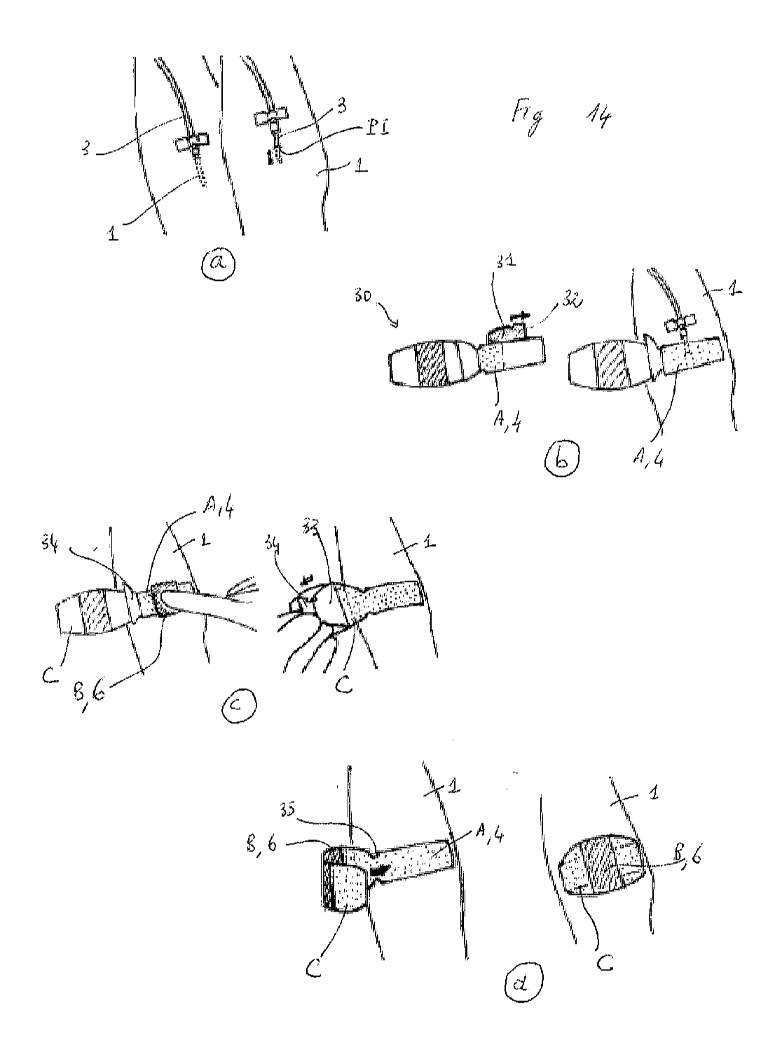

Reference is now made to FIGS. 12 and 13.

The dressing 30 represented in FIGS. 12 and 13 comprises a first part A, 4. Advantageously, a peelable strip 31 covers a first adhesive face of this first part A. A gripping tab 32 facilitates the removal of this peelable strip 31.

The dressing 30 comprises, articulated to this first part A, a third part C provided with an absorbent material B such as a compress 6. In the embodiment represented, the second part B is attached to the third part C forming the adhesive strip 7.

The first part A and the assembly formed by the second and third parts B, C are articulated about a transverse zone 35. This zone of articulation 35 may comprise a weakening line as defined previously with reference to FIGS. 7 to 11.

The compress B, 6 and the adhesive strip 7 are covered, before use, with a peelable strip 33. Advantageously, a gripping tab 34 facilitates the removal of this peelable strip 33.

In one embodiment, the compress B, 6 is at least partly surmounted by a semi-rigid and transparent sheet, for example made of polycarbonate or PET. This sheet participates in the compression of the puncture or infusion site PI, when the adhesive strip 7 is placed under tension against the compress B, 6.

Advantageously, the first part A of the dressing 30 is semi-permeable and allows the diffusion of at least certain compounds of the blood from the puncture or infusion site to the compress B, 6.

In one advantageous embodiment, the dressing, except for the compress B, 6, is made from flexible polymer material or coated fabric. The dressing, except for the compress B, 6, is advantageously entirely transparent, translucent or semi-transparent.

The flexibility of the dressing makes it possible to follow the contours of the skin, including in the vicinity of the aneurysmal zones of AVFs. Advantageously, the material constituting the third part C and where appropriate the entire dressing 30, can be extended along at least one longitudinal direction and more advantageously still along the two longitudinal and transverse directions. The dressing is thus even more adaptable to the various curves of the patient's body.

The third part C advantageously has a surface area greater than that of the first part A and is thus capable of covering it completely.

Reference is now made to FIG. 14.

Represented in FIG. 14 is the forearm 1 of a patient, a needle 3 being in place in this forearm, at a puncture or infusion site P, for example in an AVF.

The needle 3 represented is of the butterfly needle type. It is understood however that the procedure which will be described also relates to needles lacking the wings of butterfly needles, or else to catheters (for example of Cathlon® type) or else to cannulae.

In a first step, the needle 1 is advantageously slightly removed, in a manner similar to what was described with reference to FIG. 2. This slight removal of the needle 1 is symbolized by an arrow in a) of FIG. 14.

The peelable strip 31 covering the first part A, 4 of the dressing 30 is removed manually, this operation being facilitated by the presence of the gripping tab 32.

After removal of the peelable strip 31, a first face of the first part A, 4 of the dressing 30 is placed at the desired site, covering the puncture or infusion site PI, as is seen in b) of FIG. 14. This first face of the first part A, 4 of the dressing is adhesive.

Next, an absorbent material such as a compress 6 is applied with pressure against the second face of the first part A, 4 of the dressing 30, level with the puncture or infusion site PI. Advantageously, the second face of the first part of the dressing is non-adhesive.

The absorbent material may be removed after a short time, as was stated previously, so as to check the arrest of bleeding at the puncture or infusion site PI.

Next, the peelable strip 33 covering the upper face of the third part C of the dressing 30 is removed manually, this operation being facilitated by the presence of the gripping tab 34.

After removal of the peelable strip 33, a first face of the third part C of the dressing is folded about the zone of articulation 35 so as to cover the first part A, 4 of the dressing. The first face of the third part C of the dressing is adhesive. This first face is advantageously provided with an absorbent material such as a compress B, 6.

When the third part C is folded against the first part A, 4 of the dressing 30, the absorbent material B, 6 is placed, level with the puncture or infusion site PI, between the first part A and the third part C of the dressing 30.

Advantageously, the second face of the third part C of the dressing is non-adhesive.

Advantageously, the first part and the third part of the dressing comprise an adhesive strip 4 that is permeable and micro-perforated.

The perforation is advantageously produced by carrying out a process comprising the following steps:
deposition of adhesive on the face of the film that has to be made adhesive, for example a film of polyethylene (PE), especially a low-density PE film having a thickness of 60 microns, the adhesive being acrylic-based, over a thickness of around 30 microns;
deposition of a liner covering the adhesive-coated face;
perforation of the film, for example via hot needle punching, following a square mesh or lozenge mesh pattern, in one or two passes, the density of holes advantageously being of the order of 110 per square centimetre;
removal of the protective film and placement of a peelable strip.

Advantageously, the adhesive strip 4 comprises two types of perforations:
a first series of perforations, intended for the screening of the blood at the puncture site, this first series of perforations having, for example, a random or lozenge pattern;
a second series of perforations, intended to facilitate, where appropriate, the manual cutting of the adhesive strip, this second series of perforations having a square or rectangular pattern for example.

Advantageously, the perforations intended for the screening are micro-perforations obtained without removal of material, for example by needle punching.

Advantageously, the adhesive strip is elastic and the perforation is carried out with the adhesive strip under tension, so that the micropores are substantially closed after releasing the tension of the strip. During the positioning of the dressing, manual stretching of the adhesive strip causes these micropores to open.

1. Dressing for a puncture site or infusion site, comprising a first adhesive part and a second part provided with a compress, the first part being adhesive on one of its faces, a third adhesive part on one of its faces that is capable of covering the first part, the second part then being placed between the first part and the third part, the first part being intended to be attached to the skin at the puncture site or infusion site.

2. Dressing according to claim 1, characterized in that the first part is formed from a transparent, translucent or semi-transparent material.

3. Dressing according to claim 1, characterized in that the first part is made from a semi-permeable material.

4. Dressing according to claim 3, characterized in that the first part is micro-perforated.

5. Dressing according to claim 4, characterized in that the first part is formed from a strip of polyethylene provided with an acrylic adhesive.

6. Dressing according to claim 5, characterized in that the first part is provided with perforations having a diameter of less than 0.5 mm.

7. Dressing according to claim 6, characterized in that the density of perforations is of the order of 100 per square centimetre.

8. Dressing according to claim 1, characterized in that the first part extends longitudinally, the second part being articulated along an articulation zone longitudinal or transverse to the first part.

9. Dressing according to claim 1, characterized in that the third part and the first part extend substantially longitudinally and are articulated to one another.

10. Dressing according to claim 9, characterized in that the first part and the third part are articulated longitudinally or transversely to one another.

11. Dressing according to claim 10, characterized in that the third part has a length substantially equal to or greater than that of the first part.

12. Dressing according to claim 1, characterized in that at least the first part or the third part is made from a material that is elastic, in particular along the two longitudinal and/or transverse directions.

13. Dressing according to claim 1, characterized in that the third part is of substantially identical shape to the first part, for example oval, square or rectangular.

14. Infusion, drain or catheterization kit comprising a dressing as presented in claim 1 and a needle or cannula or catheter or drain.

15. Use, as an arteriovenous fistula AVF dressing, of a multilayer comprising:
a first adhesive strip that is transparent and micro-perforated;
an absorbent material;
a second adhesive strip that is articulated to the first adhesive strip (A).

16. Use according to claim 15, characterized in that:
in a first step, the first adhesive strip is placed at the desired site;
in a second step, the absorbent material is applied with pressure against the first adhesive strip;
in a third step, the vein or artery puncture needle is completely withdrawn, the pressure being maintained on the absorbent material;
in a fourth step, the absorbent material is moved away from the first adhesive strip, allowing visual control of the haemostasis;
in a fifth step, the second adhesive strip is added on top of the absorbent material.
